(19) United States
(12) Patent Application Publication
(10) Pub. No.: US 2021/0390685 A1
(43) Pub. Date: Dec. 16, 2021

(54) METHOD AND SYSTEM FOR PROVIDING CLUTTER SUPPRESSION IN VESSELS DEPICTED IN B-MODE ULTRASOUND IMAGES

(71) Applicant: GE Precision Healthcare LLC, Wauwatosa, WI (US)

(72) Inventor: Rimon Tadross, Milwaukee, WI (US)

(21) Appl. No.: 16/902,398

(22) Filed: Jun. 16, 2020

(51) Int. Cl. G06T 7/00; A61B 8/06; A61B 8/00; G06T 7/11; G06T 7/32

(52) U.S. Cl. CPC G06T 7/0012; A61B 8/06; A61B 8/461; G06T 7/11; G06T 2207/20024; G06T 2207/10132; G06T 2207/30104; G06T 2207/20084; G06T 7/32

(57) ABSTRACT

A system and method for providing clutter suppression in vessels depicted in B-mode ultrasound images is provided. The method includes acquiring series of B-mode frames and periodically acquiring a flow image frame between each series of B-mode frames. The method includes segmenting the flow image frame and a subsequent B-mode frame in a series of B-mode frames acquired immediately after the flow image frame to extract a vessel lumen and analyzing a spatial correlation between the vessel lumen region in the flow image frame and the subsequent B-mode frame. The method includes applying clutter filtering to pixels in the vessel lumen region of the subsequent B-mode frame based on flow characteristics of corresponding pixels in the flow image frame when the spatial correlation between the vessel lumen region in the flow image frame and the subsequent B-mode frame exceeds a threshold. The method includes presenting the clutter suppressed B-mode frame.

### FIELD

Certain embodiments relate to ultrasound imaging. More specifically, certain embodiments relate to a method and system for suppressing clutter seen inside blood vessels depicted in B-mode ultrasound images.

### BACKGROUND

Ultrasound imaging is a medical imaging technique for imaging organs and soft tissues in a human body. Ultrasound imaging uses real time, non-invasive high frequency sound waves to produce two-dimensional (2D), three-dimensional (3D), and/or four-dimensional (4D) (i.e., real-time/continuous 3D images) images.

Ultrasound imaging is a valuable, non-invasive tool for diagnosing various medical conditions. In an ultrasound examination of a carotid lumen or other blood vessel, a B-mode ultrasound image should appear black within the vessel lumen unless plaque is present. However, imaged tissue may cause multipath reverberations or off-axis scatterers that appear as clutter in the B-mode image, which can be problematic if the clutter appears within the vessel lumen. Currently, sonographers may rotate the probe to view the vessel lumen at a different angle to see if the appearance of the clutter/plaque changes, which may be unreliable and inefficient. Additionally and/or alternatively, sonographers may separately acquire additional flow images and review the flow images to attempt to discriminate between clutter and plaque when clutter is excessive in the B-mode image and appears similar to plaque, which may be labor intensive and inefficient.

Further limitations and disadvantages of conventional and traditional approaches will become apparent to one of skill in the art, through comparison of such systems with some aspects of the present disclosure as set forth in the remainder of the present application with reference to the drawings.

### BRIEF SUMMARY

A system and/or method is provided for providing clutter suppression in vessels depicted in B-mode ultrasound images, substantially as shown in and/or described in connection with at least one of the figures, as set forth more completely in the claims.

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a block diagram of an exemplary ultrasound system that is operable to provide clutter suppression in vessels depicted in B-mode ultrasound images, in accordance with various embodiments.

FIG. 2 is an exemplary display of a vessel depicted in a B-mode ultrasound image having clutter in the vessel lumen, in accordance with various embodiments.

FIG. 3 is an exemplary display of a vessel depicted in a B-flow ultrasound image corresponding with the B-mode ultrasound image of FIG. 2, in accordance with various embodiments.

FIG. 4 is a flow chart illustrating exemplary steps that may be utilized for providing clutter suppression in vessels depicted in B-mode ultrasound images, in accordance with exemplary embodiments.

### DETAILED DESCRIPTION

Certain embodiments may be found in a method and system for providing clutter suppression in vessels depicted in B-mode ultrasound images. Various embodiments have the technical effect of clutter filtering image pixels in a vessel lumen region of a B-mode image based on flow characteristics of corresponding image pixels in a spatially correlated flow image frame.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general-purpose signal processor or a block of random access memory, hard disk, or the like) or multiple pieces of hardware. Similarly, the programs may be stand-alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized and that structural, logical and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and preceded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional elements not having that property.

Also as used herein, the term “image” broadly refers to both viewable images and data representing a viewable image. However, many embodiments generate (or are configured to generate) at least one viewable image. In addition, as used herein, the phrase “image” is used to refer to an ultrasound mode such as B-mode (2D mode), M-mode, three-dimensional (3D) mode, CF-mode, PW Doppler, CW Doppler, MGD, and/or sub-modes of B-mode and/or CF such as Shear Wave Elasticity Imaging (SWEI), TVI, Angio, B-flow, BMI, BMI_Angio, and in some cases also MM, CM, TVD where the “image” and/or “plane” includes a single beam or multiple beams.

Furthermore, the term processor or processing unit, as used herein, refers to any type of processing unit that can carry out the required calculations needed for the various embodiments, such as single or multi-core: CPU, Accelerated Processing Unit (APU), Graphics Board, DSP, FPGA, ASIC or a combination thereof.

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and wherein the process does not form any “beams”. Also, forming of images may be performed using channel combinations that may originate from more than one transmit event (e.g., synthetic aperture techniques).

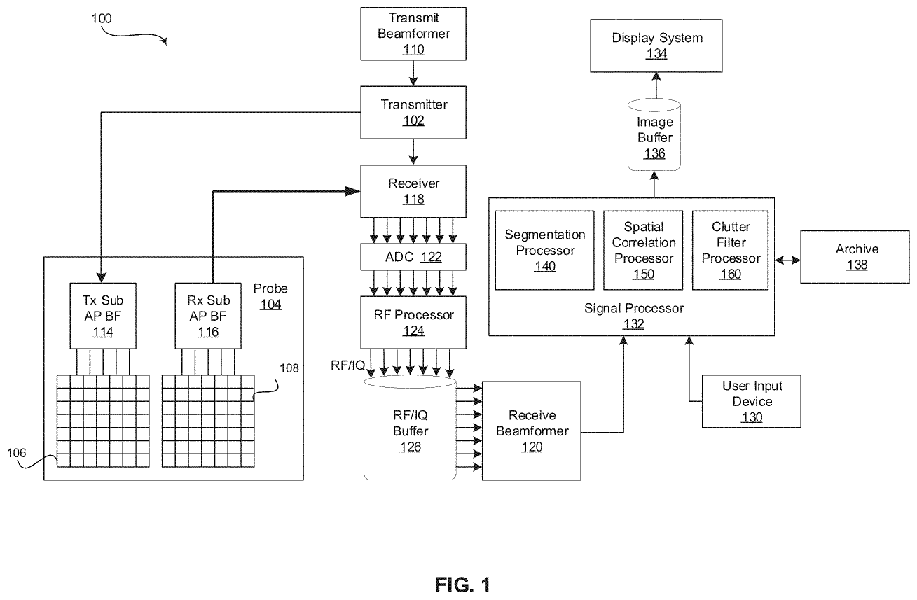

In various embodiments, ultrasound processing to form images is performed, for example, including ultrasound beamforming, such as receive beamforming, in software, firmware, hardware, or a combination thereof. One implementation of an ultrasound system having a software beamformer architecture formed in accordance with various embodiments is illustrated in FIG. 1.

FIG. 1 is a block diagram of an exemplary ultrasound system 100 that is operable to provide clutter suppression in vessels 204, 206, 304, 306 depicted in B-mode ultrasound images 202, in accordance with various embodiments. Referring to FIG. 1, there is shown an ultrasound system 100. The ultrasound system 100 comprises a transmitter 102, an ultrasound probe 104, a transmit beamformer 110, a receiver 118, a receive beamformer 120, A/D converters 122, a RF processor 124, a RF/IQ buffer 126, a user input device 130, a signal processor 132, an image buffer 136, a display system 134, and an archive 138.

The transmitter 102 may comprise suitable logic, circuitry, interfaces and/or code that may be operable to drive an ultrasound probe 104. The ultrasound probe 104 may comprise a two dimensional (2D) array of piezoelectric elements. The ultrasound probe 104 may comprise a group of transmit transducer elements 106 and a group of receive transducer elements 108, that normally constitute the same elements. In certain embodiment, the ultrasound probe 104 may be operable to acquire ultrasound image data covering at least a substantial portion of an anatomy, such as the heart, a blood vessel, or any suitable anatomical structure. In an exemplary embodiment, the ultrasound probe 104 may be operable to acquire B-mode frames with a flow image frame (e.g., B-flow, color flow, vector flow, etc.) periodically acquired between the B-mode frames. For example, a period of flow image frame acquisitions may be after every predetermined number of B-mode frames is acquired, every 0.5 second, every 0.25 seconds, or at any suitable period. In various embodiments, the period of flow frame acquisitions may be user-selectable.

The transmit beamformer 110 may comprise suitable logic, circuitry, interfaces and/or code that may be operable to control the transmitter 102 which, through a transmit sub-aperture beamformer 114, drives the group of transmit transducer elements 106 to emit ultrasonic transmit signals into a region of interest (e.g., human, animal, underground cavity, physical structure and the like). The transmitted ultrasonic signals may be back-scattered from structures in the object of interest, like blood cells or tissue, to produce echoes. The echoes are received by the receive transducer elements 108.

The group of receive transducer elements 108 in the ultrasound probe 104 may be operable to convert the received echoes into analog signals, undergo sub-aperture beamforming by a receive sub-aperture beamformer 116 and are then communicated to a receiver 118. The receiver 118 may comprise suitable logic, circuitry, interfaces and/or code that may be operable to receive the signals from the receive sub-aperture beamformer 116. The analog signals may be communicated to one or more of the plurality of A/D converters 122.

The plurality of A/D converters 122 may comprise suitable logic, circuitry, interfaces and/or code that may be operable to convert the analog signals from the receiver 118 to corresponding digital signals. The plurality of A/D converters 122 are disposed between the receiver 118 and the RF processor 124. Notwithstanding, the disclosure is not limited in this regard. Accordingly, in some embodiments, the plurality of A/D converters 122 may be integrated within the receiver 118.

The RF processor 124 may comprise suitable logic, circuitry, interfaces and/or code that may be operable to demodulate the digital signals output by the plurality of A/D converters 122. In accordance with an embodiment, the RF processor 124 may comprise a complex demodulator (not shown) that is operable to demodulate the digital signals to form I/Q data pairs that are representative of the corresponding echo signals. The RF or I/Q signal data may then be communicated to an RF/IQ buffer 126. The RF/IQ buffer 126 may comprise suitable logic, circuitry, interfaces and/or code that may be operable to provide temporary storage of the RF or I/Q signal data, which is generated by the RF processor 124.

The receive beamformer 120 may comprise suitable logic, circuitry, interfaces and/or code that may be operable to perform digital beamforming processing to, for example, sum the delayed channel signals received from RF processor 124 via the RF/IQ buffer 126 and output a beam summed signal. The resulting processed information may be the beam summed signal that is output from the receive beamformer 120 and communicated to the signal processor 132. In accordance with some embodiments, the receiver 118, the plurality of A/D converters 122, the RF processor 124, and the beamformer 120 may be integrated into a single beamformer, which may be digital. In various embodiments, the ultrasound system 100 comprises a plurality of receive beamformers 120.

The user input device 130 may be utilized to input patient data, scan parameters, settings, select protocols and/or templates, select an examination type, initiate vessel B-mode imaging functionality, select a flow frame acquisition period, and the like. In an exemplary embodiment, the user input device 130 may be operable to configure, manage and/or control operation of one or more components and/or modules in the ultrasound system 100. In this regard, the user input device 130 may be operable to configure, manage and/or control operation of the transmitter 102, the ultrasound probe 104, the transmit beamformer 110, the receiver 118, the receive beamformer 120, the RF processor 124, the RF/IQ buffer 126, the user input device 130, the signal processor 132, the image buffer 136, the display system 134, and/or the archive 138. The user input device 130 may include button(s), rotary encoder(s), a touchscreen, a touch pad, a trackball, motion tracking, voice recognition, a mousing device, keyboard, camera and/or any other device capable of receiving a user directive. In certain embodiments, one or more of the user input devices 130 may be integrated into other components, such as the display system 134, for example. As an example, user input device 130 may include a touchscreen display.

The signal processor 132 may comprise suitable logic, circuitry, interfaces and/or code that may be operable to process ultrasound scan data (i.e., summed IQ signal) for generating ultrasound images for presentation on a display system 134. The signal processor 132 is operable to perform one or more processing operations according to a plurality of selectable ultrasound modalities on the acquired ultrasound scan data. In an exemplary embodiment, the signal processor 132 may be operable to perform display processing and/or control processing, among other things. Acquired ultrasound scan data may be processed in real-time during a scanning session as the echo signals are received. Additionally or alternatively, the ultrasound scan data may be stored temporarily in the RF/IQ buffer 126 during a scanning session and processed in less than real-time in a live or off-line operation. In various embodiments, the processed image data can be presented at the display system 134 and/or may be stored at the archive 138. The archive 138 may be a local archive, a Picture Archiving and Communication System (PACS), an enterprise archive (EA), a vendor-neutral archive (VNA), or any suitable device for storing images and related information.

The signal processor 132 may be one or more central processing units, microprocessors, microcontrollers, and/or the like. The signal processor 132 may be an integrated component, or may be distributed across various locations, for example. In an exemplary embodiment, the signal processor 132 may comprise a segmentation processor 140, a spatial correlation processor 150, and a clutter filter processor 160. The signal processor 132 may be capable of receiving input information from a user input device 130 and/or archive 138, receiving image data, generating an output displayable by a display system 134, and manipulating the output in response to input information from a user input device 130, among other things. The signal processor 132, including the segmentation processor 140, the spatial correlation processor 150, and the clutter filter processor 160, may be capable of executing any of the method(s) and/or set(s) of instructions discussed herein in accordance with the various embodiments, for example.

The ultrasound system 100 may be operable to continuously acquire ultrasound scan data at a frame rate that is suitable for the imaging situation in question. Typical frame rates range from 20-120 but may be lower or higher. The acquired ultrasound scan data may be displayed on the display system 134 at a display-rate that can be the same as the frame rate, or slower or faster. An image buffer 136 is included for storing processed frames of acquired ultrasound scan data that are not scheduled to be displayed immediately. Preferably, the image buffer 136 is of sufficient capacity to store at least several minutes' worth of frames of ultrasound scan data. The frames of ultrasound scan data are stored in a manner to facilitate retrieval thereof according to its order or time of acquisition. The image buffer 136 may be embodied as any known data storage medium.

The signal processor 132 may include a segmentation processor 140 that comprises suitable logic, circuitry, interfaces and/or code that may be operable to segment flow image frames and B-mode frames. The segmentation processor 140 may be used to extract a vessel lumen region, such as a carotid lumen region, in flow images and B-mode frames. In this regard, the segmentation processor 140 may include, for example, artificial intelligence image analysis algorithms, one or more deep neural networks (e.g., a convolutional neural network such as u-net) and/or may utilize any suitable form of artificial intelligence image analysis techniques or machine learning processing functionality configured to provide automated segmentation functionality. Additionally and/or alternatively, the artificial intelligence image analysis techniques or machine learning processing functionality configured to provide the automated segmentation may be provided by a different processor or distributed across multiple processors at the ultrasound system 100 and/or a remote processor communicatively coupled to the ultrasound system 100. For example, the image segmentation functionality may be provided as a deep neural network that may be made up of, for example, an input layer, an output layer, and one or more hidden layers in between the input and output layers. Each of the layers may be made up of a plurality of processing nodes that may be referred to as neurons. For example, the image segmentation functionality may include an input layer having a neuron for each pixel or a group of pixels from a B-mode frame or flow image frame of an anatomical structure. The output layer may have a neuron corresponding to a plurality of pre-defined anatomical structures, such as a vessel lumen region or any suitable anatomical structure. Each neuron of each layer may perform a processing function and pass the processed B-mode or flow image information to one of a plurality of neurons of a downstream layer for further processing. As an example, neurons of a first layer may learn to recognize edges of structure in the B-mode and flow image frames. The neurons of a second layer may learn to recognize shapes based on the detected edges from the first layer. The neurons of a third layer may learn positions of the recognized shapes relative to landmarks in the B-mode and flow image frames. The processing performed by the deep neural network may identify anatomical structures and the location of the structures in the B-mode and flow image frames with a high degree of probability.

In an exemplary embodiment, the segmentation processor 140 may be configured to store the image segmentation information at archive 138 and/or any suitable storage medium. The image segmentation information may be provided to the spatial correlation processor 150 for analyzing a spatial correlation between a vessel lumen region in a flow image frame and a B-mode frame, as described below.

The signal processor 132 may include a spatial correlation processor 150 that comprises suitable logic, circuitry, interfaces and/or code that may be operable to analyze a spatial correlation between a vessel lumen region 304 in a flow image frame 302 and a subsequent B-mode frame 202. For example, the ultrasound system 100 may acquire a plurality of series of B-mode frames, each of the series of B-mode frames comprising a plurality of B-mode frames 202. The ultrasound system 100 may periodically acquire a flow image frame 302 between each of the plurality of series of B-mode frames 202. The B-mode frames 202 and flow image frames 302 may be segmented by the segmentation processor 140 as described above to extract a vessel lumen region 204, 304 in the B-mode frames 202 and flow image frame 302. The spatial correlation processor 150 is configured to determine whether the sonographer is holding the probe substantially stationary to examine an area of interest or moving the probe to identify a desired plane. If the sonographer is not moving the probe or moving the probe slowly to examine an area of interest, the flow image frame 302 and subsequently acquired B-mode frames 202 may be spatially correlated. If the sonographer is moving the probe 104 to identify a desired plane, the flow image frame 302 and subsequently acquired B-mode frames 202 may not be spatially correlated. The spatial correlation processor 150 may be configured to analyze a spatial correlation between each of the acquired flow frames 302 and a subsequent B-mode frame 202 (and each subsequent B-mode frame 202 until the next flow frame 302) to generate a correlation coefficient associated with each B-mode frame 202. The spatial correlation processor 150 may initiate clutter suppression in the B-mode frame(s) 202 when the correlation coefficient exceeds a threshold. The B-mode frame(s) 202 may be presented at the display system 134 without clutter suppression when the correlation coefficient does not exceed the threshold. In this way, the B-mode frame(s) 202 spatially correlated to the previously acquired flow image frame 302 are clutter filtered while the B-mode frame(s) not spatially correlated to the previously acquired flow image frame 302 are presented without clutter filtering.

The signal processor 132 may include a clutter filter processor 160 that comprises suitable logic, circuitry, interfaces and/or code that may be operable to apply clutter filtering to image pixels 208 in a vessel lumen region 204 of a B-mode frame 202 based on flow characteristics of corresponding image pixels 308 in a vessel lumen region 304 of a flow image frame 302. For example, when the correlation coefficient of a B-mode frame 202 exceeds the correlation coefficient as determined by the spatial correlation processor 150, the clutter filter processor 160 provides suppression to image pixels in the vessel lumen region 204 of the B-mode frame 202 that correspond to image pixels in the vessel lumen region 304 of the previously acquired flow image frame 302 based on the flow characteristics of the image pixels in the vessel lumen region 304 of the flow image frame 302. As an example, image pixels in the B-mode frame 202 corresponding to image pixels in the flow image frame 302 with no or low flow may not be suppressed, whereas image pixels in the B-mode frame 202 corresponding to image pixels in the flow image frame 302 having flow are suppressed. In various embodiments, an amount of suppression is smoothly applied by the clutter filter processor 160 based on an amount of flow. For example, the amount of suppression may be provided on a sliding scale based on a degree of flow, with no or low flow providing no or low suppression, some flow providing some suppression, and high flow providing full suppression. The clutter filtered B-mode frame may be presented at the display system 134 and/or stored at archive 138 or any suitable data storage medium. The processing and presentation of the clutter filtered B-mode frame may obviate the need for a sonographer to rotate the probe 104 to view the vessel lumen 204 at a different angle to see if the appearance of the clutter/plaque changes and/or separately review flow images to attempt to discriminate between clutter and plaque when clutter is excessive in the B-mode frame 202 and appears similar to plaque.

The display system 134 may be any device capable of communicating visual information to a user. For example, a display system 134 may include a liquid crystal display, a light emitting diode display, and/or any suitable display or displays. The display system 134 can be operable to display information from the signal processor 132 and/or archive 138, such as B-mode image frames with and/or without clutter filtering and/or any suitable information.

The archive 138 may be one or more computer-readable memories integrated with the ultrasound system 100 and/or communicatively coupled (e.g., over a network) to the ultrasound system 100, such as a Picture Archiving and Communication System (PACS), an enterprise archive (EA), a vendor-neutral archive (VNA), a server, a hard disk, floppy disk, CD, CD-ROM, DVD, compact storage, flash memory, random access memory, read-only memory, electrically erasable and programmable read-only memory and/or any suitable memory. The archive 138 may include databases, libraries, sets of information, or other storage accessed by and/or incorporated with the signal processor 132, for example. The archive 138 may be able to store data temporarily or permanently, for example. The archive 138 may be capable of storing medical image data, data generated by the signal processor 132, and/or instructions readable by the signal processor 132, among other things. In various embodiments, the archive 138 stores B-mode frames 202, flow image frames 302, segmentation information generated by the segmentation processor 140, instructions for performing image segmentation, instructions for performing spatial correlation, a spatial correlation threshold, and/or instructions for performing clutter filtering, among other things.

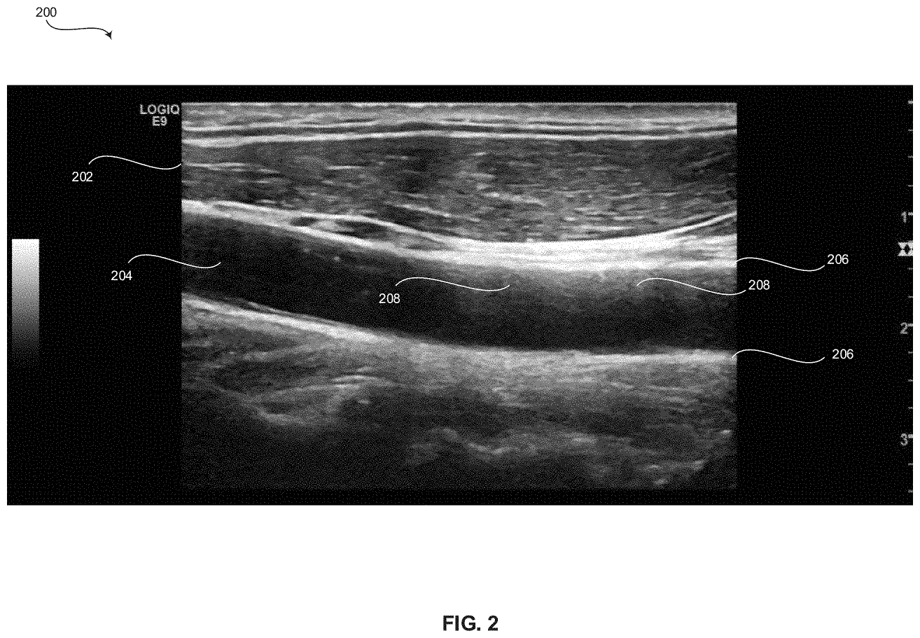

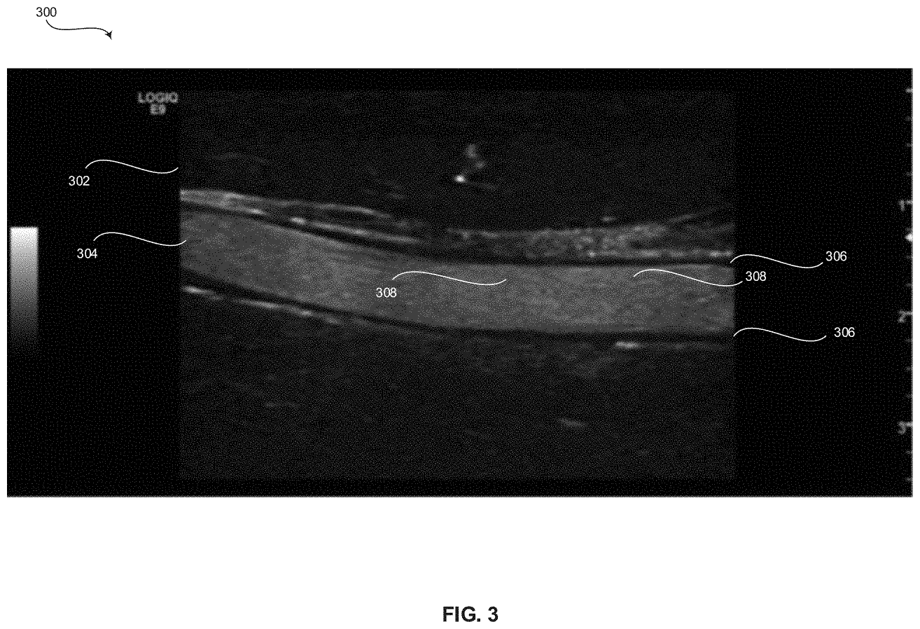

FIG. 2 is an exemplary display 200 of a vessel 204, 206 depicted in a B-mode ultrasound image 202 having clutter 208 in the vessel lumen 204, in accordance with various embodiments. Referring to FIG. 3, the display 200 presents a B-mode frame 202 depicting a vessel 204, 206 having a vessel lumen 204 and vessel walls 206. The vessel lumen region 204 includes an area of pixels 208 depicting either clutter or plaque. The B-mode frame 202 may be presented at a display 200 of a display system 134 and/or may be stored at archive 138 or any suitable data storage medium.

FIG. 3 is an exemplary display 300 of a vessel 304, 306 depicted in a B-flow ultrasound image 302 corresponding with the B-mode ultrasound image 202 of FIG. 2, in accordance with various embodiments. Referring to FIG. 3, the display 300 presents a flow image frame, such as a B-flow image frame 302, color flow image, frame a vector flow image frame, or the like that corresponds with the B-mode frame 202 of FIG. 2. B-flow imaging is described, for example, in Berger et al., “B-Flow Technology,” GE Healthcare, http://www3.gehealthcare.n1/˜/media/downloads/uk/product/ultrasound/logiq/logiq %20xdclear% 20family/white%20paper%20-%20b-flow%20technology% 20-%20playbuttons.pdf?Parent=%7B26474D70-5245-4B95-96DC-207455743EC2%7D, 2012, which is incorporated by reference herein in its entirety. The flow image frame 302 depicts a vessel 304, 306 having a vessel lumen 304 and vessel walls 306. The vessel lumen 304 does not show plaque in the image pixel areas 308 of the lumen 304 that included clutter/plaque 208 in the B-mode frame 202. Accordingly, the clutter/plaque image pixel areas 208 in the vessel lumen region 204 of FIG. 2 is clutter as opposed to plaque. In a representative embodiment, the flow image frame 302 is segmented to extract the vessel lumen region 304 and the vessel lumen region 304 is spatially correlated to a vessel lumen region 204 of a segmented B-mode frame 202. In various embodiments, if the spatial correlation between the vessel lumen regions 204, 304 of the B-mode image 202 and the flow image frame 302 exceed a correlation threshold, the flow characteristics of the image pixels 308 in the vessel lumen 304 of the flow image 302 may be used to perform clutter filtering on the image pixels 208 in the vessel lumen region 204 of the B-mode image 202. In an exemplary embodiment, the flow image 302 may not be presented at a display 300 of a display system 134. Instead, the periodically acquired flow image 302 may be used by a clutter filter processor 160 to apply clutter filtering to a subsequently acquired B-mode frame 202, which may be presented at a display 200 of the display system 134. The flow image frame 302 may be stored at archive 138 or any suitable data storage medium.

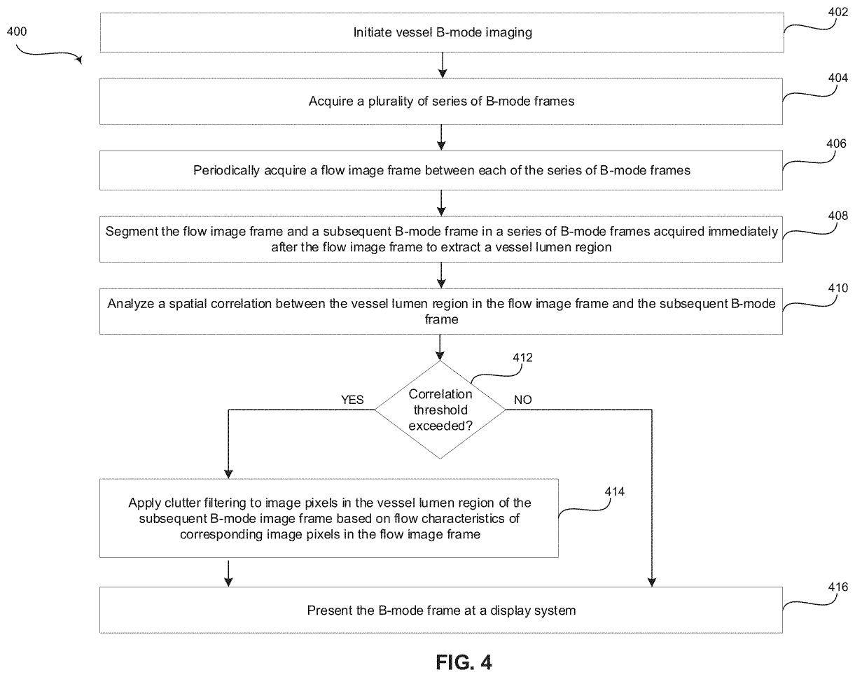

FIG. 4 is a flow chart 400 illustrating exemplary steps 402-416 that may be utilized for providing clutter suppression in vessels 204, 206 depicted in B-mode ultrasound images 202, in accordance with exemplary embodiments. Referring to FIG. 4, there is shown a flow chart 400 comprising exemplary steps 402 through 416. Certain embodiments may omit one or more of the steps, and/or perform the steps in a different order than the order listed, and/or combine certain of the steps discussed below. For example, some steps may not be performed in certain embodiments. As a further example, certain steps may be performed in a different temporal order, including simultaneously, than listed below.

At step 402, an ultrasound system 100 may initiate vessel (e.g., carotid) B-mode imaging. For example, the vessel B-mode imaging may be a default setting, a mode selection, an examination type selection (e.g., carotid ultrasound image examination), or any suitable user selection. In various embodiments, a flow frame acquisition period may be user-definable. As an example, a sonographer may define, via the user input device 130, the flow frame acquisition period to be after a selectable number of B-mode frame acquisitions, every 0.5 seconds, every 0.25 seconds, and/or any suitable acquisition period.

At step 404, the ultrasound system 100 may acquire a plurality of series of B-mode frames 202. For example, the ultrasound probe 104 may acquire multiple series of B-mode frames 202, each series having a plurality of B-mode frames, and provide the acquired B-mode frames 202 to the signal processor 132. The number of B-mode frames 202 in each series of B-mode frames 202 may be based on the flow frame acquisition period, which may be a default period or a user-defined period selected at step 402.

At step 406, the ultrasound system 100 may periodically acquire a flow image frame 302 between each of the series of B-mode frames 202. For example, the ultrasound probe 104 may acquire a flow image frame 302, such as a B-flow image frame, a color flow image frame, a vector flow image frame, and/or any suitable flow image frame, between each series of B-mode frames 202 and provide the flow image frames 302 to the signal processor 132. The flow frame acquisition period may be a default period or a user-defined period selected at step 402. In various embodiments, the flow image frames 302 are not presented at the display system 134.

At step 408, a signal processor 132 of the ultrasound system 100 may segment the flow image frame 302 and a subsequent B-mode frame 202 in a series of B-mode frames acquired immediately after the flow image frame 302 to extract a vessel lumen region 204, 304. For example, a segmentation processor 140 of the signal processor 132 may be configured to segment flow image frames 302 and B-mode frames 202. The segmentation processor 140 may be used to extract a vessel lumen region 204, 304, such as a carotid lumen region, in flow images 302 and B-mode frames 202. The segmentation processor 140 may include and/or may be communicatively coupled to one or more processor that include, for example, artificial intelligence image analysis algorithms, one or more deep neural networks (e.g., a convolutional neural network such as u-net) and/or may utilize any suitable form of artificial intelligence image analysis techniques or machine learning processing functionality configured to provide automated segmentation functionality.

At step 410, the signal processor 132 of the ultrasound system 100 may analyze a spatial correlation between the vessel lumen region 204, 304 in the flow image frame 302 and the subsequent B-mode frame 202. For example, a spatial correlation processor 150 of the signal processor may be configured to determine whether the sonographer is holding the ultrasound probe 104 substantially stationary to examine an area of interest or moving the probe to identify a desired plane. The spatial correlation processor 150 may be configured to analyze a spatial correlation between each of the acquired flow frames 302 and a subsequent B-mode frame 202 (and each subsequent B-mode frame 202 until the next flow frame 302) to generate a correlation coefficient associated with each B-mode frame 202.

At step 412, the signal processor 132 of the ultrasound system 100 determines whether a correlation threshold has been exceeded. For example, the spatial correlation processor 150 may initiate clutter suppression in the B-mode frame(s) 202 by proceeding to step 414 when the correlation coefficient exceeds a threshold. The process may forego step 414 and proceed to step 416 to display the B-mode frame(s) 202 at the display system 134 without clutter suppression when the correlation coefficient does not exceed the threshold. Accordingly, the B-mode frame(s) 202 spatially correlated to the previously acquired flow image frame 302 are clutter filtered while the B-mode frame(s) not spatially correlated to the previously acquired flow image frame 302 are presented without clutter filtering based on the determination of whether the correlation coefficient exceeds the threshold at step 412.

At step 414, the signal processor 132 of the ultrasound system 100 may apply clutter filtering to image pixels 208 in the vessel lumen region 204 of the subsequent B-mode image frame 202 based on the flow characteristics of corresponding image pixels 308 in the flow image frame 302. For example, when the correlation coefficient of a B-mode frame 202 exceeds the correlation coefficient as determined by the spatial correlation processor 150 at step 412, a clutter filter processor 160 of the signal processor 132 provides suppression to image pixels 208 in the vessel lumen region 204 of the B-mode frame 202 that correspond to image pixels 308 in the vessel lumen region 304 of the previously acquired flow image frame 302 based on the flow characteristics of the image pixels 308 in the vessel lumen region 304 of the flow image frame 302. As an example, image pixels 208 in the B-mode frame 202 corresponding to image pixels 308 in the flow image frame 302 with no or low flow may not be suppressed, whereas image pixels 208 in the B-mode frame 202 corresponding to image pixels 308 in the flow image frame 302 having flow are suppressed. In various embodiments, an amount of suppression is smoothly applied by the clutter filter processor 160 based on an amount of flow, such as no or low suppression provided for no or low flow, some suppression provided for some flow, and full suppression provided for high flow.

At step 416, the signal processor 132 of the ultrasound system 100 may present the B-mode frame 202 at a display system 134. The B-mode frame 202 may be presented with the clutter filtering performed at step 414 or without clutter filtering based on whether the correlation threshold is exceeded as determined at step 412.

Aspects of the present disclosure provide clutter suppression in vessels 204, 206 depicted in B-mode ultrasound images 200. In accordance with various embodiments, the method 400 may comprise acquiring 404, by an ultrasound probe 104 of an ultrasound system 100, a plurality of series of B-mode frames, each of the plurality of series of B-mode frames comprising a plurality of B-mode frames 202. The method 400 may comprise periodically acquiring 406, by the ultrasound probe 104, a flow image frame 302 between each of the plurality of series of B-mode frames 202. The method 400 may comprise segmenting 408, by at least one processor 132, 140 of the ultrasound system 100, the flow image frame 302 and a subsequent B-mode frame 202 in a series of B-mode frames acquired immediately after the flow image frame 302 to extract a vessel lumen region 204, 304 in each of the flow image frame 302 and the subsequent B-mode frame 202. The method 400 may comprise analyzing 410, by the at least one processor 132, 150, a spatial correlation between the vessel lumen region 204, 304 in the flow image frame 302 and the subsequent B-mode frame 202. The method 400 may comprise applying 414, by the at least one processor 132, 160, clutter filtering to image pixels 208 in the vessel lumen region 204 of the subsequent B-mode frame 202 to generate a clutter suppressed B-mode frame based on flow characteristics of corresponding image pixels 308 in the flow image frame 302 when 412 the spatial correlation between the vessel lumen region 204, 304 in the flow image frame 302 and the subsequent B-mode frame 202 exceeds a threshold. The method 400 may comprise presenting 416, at a display system 134 of the ultrasound system 100, the clutter suppressed B-mode frame.

In a representative embodiment, the flow image frame 302 is a B-flow image frame. In an exemplary embodiment, the flow image frame 302 is one of a color flow image frame or a vector flow image frame. In certain embodiments, the vessel lumen region 204, 304 is a lumen region of a carotid artery. In various embodiments, the method 400 may comprise presenting 416, at the display system 134, the subsequent B-mode frame 202 in a series of B-mode frames acquired immediately after the flow image frame 302 without the applying 414 the clutter filtering when 412 the spatial correlation between the vessel lumen region 204, 304 in the flow image frame 302 and the subsequent B-mode frame 202 does not exceed the threshold. In a representative embodiment, the analyzing 410 the spatial correlation may generate a correlation coefficient. The threshold may be a threshold correlation coefficient. In an exemplary embodiment, the segmenting 408 the flow image frame and the subsequent B-mode frame 202 to extract the vessel lumen region 204 is performed by the at least one processor executing artificial intelligence. In certain embodiments, the applying 414 the clutter filtering is smoothly applied by the at least one processor 132, 160 to each of the image pixels 208 in the vessel lumen region 204 of the subsequent B-mode frame 202 based on an amount of flow defined by the flow characteristics of the corresponding image pixels 308 in the flow image frame 302.

Various embodiments provide an ultrasound system 100 for providing clutter suppression in vessels 204, 206 depicted in B-mode ultrasound images 202. The ultrasound system 100 may comprise an ultrasound probe 104, at least one processor 132, 140, 150, 160, and a display system 134. The ultrasound probe 104 may be configured to acquire a plurality of series of B-mode frames, each of the plurality of series of B-mode frames comprising a plurality of B-mode frames 202. The ultrasound probe 104 may be configured to periodically acquire a flow image frame 302 between each of the plurality of series of B-mode frames 202. The at least one processor 132, 140 may be configured to segment the flow image frame 302 and a subsequent B-mode frame 202 in a series of B-mode frames acquired immediately after the flow image frame 302 to extract a vessel lumen region 204, 304 in each of the flow image frame 302 and the subsequent B-mode frame 202. The at least one processor 132, 150 may be configured to analyze a spatial correlation between the vessel lumen region 204, 304 in the flow image frame 302 and the subsequent B-mode frame 202. The at least one processor 132, 160 may be configured to apply clutter filtering to image pixels 208 in the vessel lumen region 204 of the subsequent B-mode frame 202 to generate a clutter suppressed B-mode frame based on flow characteristics of corresponding image pixels 308 in the flow image frame 302 when the spatial correlation between the vessel lumen region 204, 304 in the flow image frame 302 and the subsequent B-mode frame 202 exceeds a threshold. The display system 134 may be configured to present the clutter suppressed B-mode frame.

In an exemplary embodiment, the flow image frame 302 is a B-flow image frame. In certain embodiments, the flow image frame 302 is one of a color flow image frame or a vector flow image frame. In various embodiments, the subsequent B-mode frame 202 in a series of B-mode frames acquired immediately after the flow image frame 302 may be presented at the display system 134 without the at least one processor 132, 160 applying the clutter filtering when the spatial correlation between the vessel lumen region 204, 304 in the flow image frame 302 and the subsequent B-mode frame 202 does not exceed the threshold. In a representative embodiment, the at least one processor 132, 150 generates a correlation coefficient based on the spatial correlation analysis. The threshold is a threshold correlation coefficient. In certain embodiments, the at least one processor 132, 140 executes artificial intelligence to segment the flow image frame 302 and the subsequent B-mode frame 202 to extract the vessel lumen region 204, 304. In an exemplary embodiment, the at least one processor 132, 160 may be configured to smoothly apply the clutter filtering to each of the image pixels 208 in the vessel lumen region 204 of the subsequent B-mode frame 202 based on an amount of flow defined by the flow characteristics of the corresponding image pixels 308 in the flow image frame 302.

Certain embodiments provide a non-transitory computer readable medium having stored thereon, a computer program having at least one code section. The at least one code section is executable by a machine for causing an ultrasound system to perform steps 400. The steps 400 may comprise acquiring 404 a plurality of series of B-mode frames, each of the plurality of series of B-mode frames comprising a plurality of B-mode frames 202. The steps 400 may comprise periodically acquiring 406 a flow image frame 302 between each of the plurality of series of B-mode frames 202. The steps 400 may comprise segmenting 408 the flow image frame 302 and a subsequent B-mode frame 202 in a series of B-mode frames acquired immediately after the flow image frame 302 to extract a vessel lumen region 204, 304 in each of the flow image frame 302 and the subsequent B-mode frame 202. The steps 400 may comprise analyzing 410 a spatial correlation between the vessel lumen region 204, 304 in the flow image frame 302 and the subsequent B-mode frame 202. The steps 400 may comprise applying 414 clutter filtering to image pixels 208 in the vessel lumen region 204 of the subsequent B-mode frame 202 to generate a clutter suppressed B-mode frame based on flow characteristics of corresponding image pixels 308 in the flow image frame 302 when 412 the spatial correlation between the vessel lumen region 204, 304 in the flow image frame 302 and the subsequent B-mode frame 202 exceeds a threshold. The steps 400 may comprise presenting 416 the clutter suppressed B-mode frame at a display system 134 of the ultrasound system 100.

In various embodiments, the flow image frame 302 is a B-flow image frame. In a representative embodiment, the steps 400 may comprise presenting 416 the subsequent B-mode frame 202 in a series of B-mode frames acquired immediately after the flow image frame 302 at the display system 134 without the applying 414 the clutter filtering when 412 the spatial correlation between the vessel lumen region 204, 304 in the flow image frame 302 and the subsequent B-mode frame 202 does not exceed the threshold. In an exemplary embodiment, the segmenting 408 the flow image frame 302 and the subsequent B-mode frame 202 to extract the vessel lumen region 204, 304 may be performed by artificial intelligence. In certain embodiments, the applying 414 the clutter filtering is smoothly applied to each of the image pixels 208 in the vessel lumen region 204 of the subsequent B-mode frame 202 based on an amount of flow defined by the flow characteristics of the corresponding image pixels 308 in the flow image frame 302.

As utilized herein the term “circuitry” refers to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” or “configured” to perform a function whenever the circuitry comprises the necessary hardware and code (if any is necessary) to perform the function, regardless of whether performance of the function is disabled, or not enabled, by some user-configurable setting.

Other embodiments may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein for providing clutter suppression in vessels depicted in B-mode ultrasound images.

Accordingly, the present disclosure may be realized in hardware, software, or a combination of hardware and software. The present disclosure may be realized in a centralized fashion in at least one computer system, or in a distributed fashion where different elements are spread across several interconnected computer systems. Any kind of computer system or other apparatus adapted for carrying out the methods described herein is suited.

Various embodiments may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

What is claimed is:

1. A method comprising:
acquiring, by an ultrasound probe of an ultrasound system, a plurality of series of B-mode frames, each of the plurality of series of B-mode frames comprising a plurality of B-mode frames;
periodically acquiring, by the ultrasound probe, a flow image frame between each of the plurality of series of B-mode frames;
segmenting, by at least one processor of the ultrasound system, the flow image frame and a subsequent B-mode frame in a series of B-mode frames acquired immediately after the flow image frame to extract a vessel lumen region in each of the flow image frame and the subsequent B-mode frame;
analyzing, by the at least one processor, a spatial correlation between the vessel lumen region in the flow image frame and the subsequent B-mode frame;
applying, by the at least one processor, clutter filtering to image pixels in the vessel lumen region of the subsequent B-mode frame to generate a clutter suppressed B-mode frame based on flow characteristics of corresponding image pixels in the flow image frame when the spatial correlation between the vessel lumen region in the flow image frame and the subsequent B-mode frame exceeds a threshold; and
presenting, at a display system of the ultrasound system, the clutter suppressed B-mode frame.

2. The method of claim 1, wherein the flow image frame is a B-flow image frame.

3. The method of claim 1, wherein the flow image frame is one of a color flow image frame or a vector flow image frame.

4. The method of claim 1, wherein the vessel lumen region is a lumen region of a carotid artery.

5. The method of claim 1, comprising presenting, at the display system, the subsequent B-mode frame in a series of B-mode frames acquired immediately after the flow image frame without the applying the clutter filtering when the spatial correlation between the vessel lumen region in the flow image frame and the subsequent B-mode frame does not exceed the threshold.

6. The method of claim 1, wherein the analyzing the spatial correlation generates a correlation coefficient, and wherein the threshold is a threshold correlation coefficient.

7. The method of claim 1, wherein the segmenting the flow image frame and the subsequent B-mode frame to extract the vessel lumen region is performed by the at least one processor executing artificial intelligence.

8. The method of claim 1, wherein the applying the clutter filtering is smoothly applied by the at least one processor to each of the image pixels in the vessel lumen region of the subsequent B-mode frame based on an amount of flow defined by the flow characteristics of the corresponding image pixels in the flow image frame.

9. An ultrasound system comprising:
an ultrasound probe operable to:
acquire a plurality of series of B-mode frames, each of the plurality of series of B-mode frames comprising a plurality of B-mode frames; and
periodically acquire a flow image frame between each of the plurality of series of B-mode frames;
at least one processor configured to:
segment the flow image frame and a subsequent B-mode frame in a series of B-mode frames acquired immediately after the flow image frame to extract a vessel lumen region in each of the flow image frame and the subsequent B-mode frame;
analyze a spatial correlation between the vessel lumen region in the flow image frame and the subsequent B-mode frame; and
apply clutter filtering to image pixels in the vessel lumen region of the subsequent B-mode frame to generate a clutter suppressed B-mode frame based on flow characteristics of corresponding image pixels in the flow image frame when the spatial correlation between the vessel lumen region in the flow image frame and the subsequent B-mode frame exceeds a threshold; and
a display system configured to present the clutter suppressed B-mode frame.

10. The system of claim 9, wherein the flow image frame is a B-flow image frame.

11. The system of claim 9, wherein the flow image frame is one of a color flow image frame or a vector flow image frame.

12. The system of claim 9, wherein the subsequent B-mode frame in a series of B-mode frames acquired immediately after the flow image frame is presented at the display system without the at least one processor applying the clutter filtering when the spatial correlation between the vessel lumen region in the flow image frame and the subsequent B-mode frame does not exceed the threshold.

13. The system of claim 9, wherein the at least one processor generates a correlation coefficient based on the spatial correlation analysis, and wherein the threshold is a threshold correlation coefficient.

14. The system of claim 9, wherein the at least one processor executes artificial intelligence to segment the flow image frame and the subsequent B-mode frame to extract the vessel lumen region.

15. The system of claim 9, wherein the at least one processor is configured to smoothly apply the clutter filtering to each of the image pixels in the vessel lumen region of the subsequent B-mode frame based on an amount of flow defined by the flow characteristics of the corresponding image pixels in the flow image frame.

16. A non-transitory computer readable medium having stored thereon, a computer program having at least one code section, the at least one code section being executable by a machine for causing an ultrasound system to perform steps comprising:
acquiring a plurality of series of B-mode frames, each of the plurality of series of B-mode frames comprising a plurality of B-mode frames;
periodically acquiring a flow image frame between each of the plurality of series of B-mode frames;
segmenting the flow image frame and a subsequent B-mode frame in a series of B-mode frames acquired immediately after the flow image frame to extract a vessel lumen region in each of the flow image frame and the subsequent B-mode frame;
analyzing a spatial correlation between the vessel lumen region in the flow image frame and the subsequent B-mode frame;
applying clutter filtering to image pixels in the vessel lumen region of the subsequent B-mode frame to generate a clutter suppressed B-mode frame based on flow characteristics of corresponding image pixels in the flow image frame when the spatial correlation between the vessel lumen region in the flow image frame and the subsequent B-mode frame exceeds a threshold; and
presenting the clutter suppressed B-mode frame at a display system of the ultrasound system.

17. The non-transitory computer readable medium of claim 16, wherein the flow image frame is a B-flow image frame.

18. The non-transitory computer readable medium of claim 16, comprising presenting the subsequent B-mode frame in a series of B-mode frames acquired immediately after the flow image frame at the display system without the applying the clutter filtering when the spatial correlation between the vessel lumen region in the flow image frame and the subsequent B-mode frame does not exceed the threshold.

19. The non-transitory computer readable medium of claim 16, wherein the segmenting the flow image frame and the subsequent B-mode frame to extract the vessel lumen region is performed by artificial intelligence.

20. The non-transitory computer readable medium of claim 16, wherein the applying the clutter filtering is smoothly applied to each of the image pixels in the vessel lumen region of the subsequent B-mode frame based on an amount of flow defined by the flow characteristics of the corresponding image pixels in the flow image frame.
